(19) United States
(12) Patent Application Publication
(10) Pub. No.: US 2003/0194450 A1
(43) Pub. Date: Oct. 16, 2003

(54) Method of producing a preparation containing active substances from cannabis

(76) Inventor: Ulrich Thomet, Bern (CH)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/410,800

(22) Filed: Apr. 10, 2003

(30) Foreign Application Priority Data
Apr. 10, 2002 (EP) 02405284.7

(51) Int. Cl. A61K 35/78

(52) U.S. Cl. 424/725; 424/774

(57) ABSTRACT

Milk and milk products are produced having a cannabis content. These products are generated in milk cows fed at least partially with hemp plant parts, in particular blossom material of pistillate hemp plants, these hemp plant parts being administered as silage fodder, dried fodder or fresh fodder mixed with usual cattle feed. The milk from cows so fed is obtained and processed into a milk product through a processing step of milk processing technology, which product can in itself be used, or can be used as a component in another product with pharmacological properties.

### CROSS-REFERENCE TO RELATED APPLICATIONS

This application claims the benefit of priority under 35 U.S.C. §119 to European Patent Application Number 02,405,284.7, filed Apr. 10, 2002.

### BACKGROUND OF THE INVENTION

1. Field of the Invention

This invention relates to a preparation containing active ingredients from the cannabinoid class of active substances. In particular it relates to the production of a milk which contains cannabinoid active substances and to products obtained from this milk. The method comprises essentially natural biological processes, a product being obtained which has been subjected to neither chemical processes nor extraction processes with solvents.

2. Background

Products obtained from hemp species, such as resin or dried leaves, blossoms and buds, have been used for centuries as remedies, as preparations for cults and rituals in Asian cultures, as medicines and as narcotic drugs. The most important hemp variety is cannabis sativa, which occurs primarily in North and South America. In addition there are also other hemp species, including Asian hemp varieties such as cannabis indica or cannabis ruderalis, and others which may occur as pistillate plants. Known as the main active substance in these and other hemp species is tetrahydrocannabinol (THC, e.g., Δ-9-tetrahydrocannabinol). This substance is used as the active ingredient in numerous pharmaceutical products. The active substance is reported to have neither clearly narcotizing nor clearly stimulating effects. THC is reported to have an effect on the serotonin level, whereby nervous conditions are relaxed. THC is also said to stimulate certain cerebral functions and possess hallucinogenic properties. THC is reported to be massively stored in the body and thus can be shown to exist in the body as a metabolic product even a long time after ingestion. In Chinese medicine the hemp active substance has been used to treat rheumatism, malaria and constipation. Use of hemp has also been documented in India for several hundred years, as a remedy and drug, primarily for psychosomatic diseases.

Pharmaceutical preparations which contain extracts from cannabis can also be found in patent literature. For example, described in the German patent DE 4403157A1, or the corresponding British patent GB 2274588A, is a pain-relieving preparation for topical application on the skin containing, among other things, an extract of cannabis sativa (see Example 7 therein). The active substances are obtained from the plants by means of extraction. Described in the German patent DE 19757921A1 is a preparation for activating hair growth in which dried plant parts of cannabis sativa are used.

There are numerous areas of application in which pharmaceutical compounds containing cannabis can be used: glaucoma, diseases connected with lack of appetite, nausea, and nausea during chemotherapy, stress, migraines, depression, asthma, epilepsy, spasms. A further possible application is the use of pharmaceutical cannabis as a detoxicant for persons contaminated by certain poisons released into the environment.

The potential useful applications for products of this kind are manifold, aside from the generation of a state of intoxication. In addition to the use of plant components (e.g., leaves, blossoms or portions thereof, stems, seeds, etc.), resin and plant extracts of cannabis are also used. Cannabis is also smoked as a means of administering the active substance (e.g., THC), although delivery by this route is not without certain undesirable effects (e.g., deleterious effects to airways, inefficiencies in monitoring dosage, second-hand smoke, etc.). Clearly there is a need for dosage-controlled, optimal and well tolerated forms of administration of cannabis active substances, on the one hand in order to exploit a wide range of active ingredients, and, on the other hand, to avoid a harmful form of administration. The present invention addresses such a need and offers other related advantages.

### SUMMARY OF THE INVENTION

According to the present invention there is provided a method of producing a milk product that contains at least one cannabinoid active substance, comprising feeding a dairy animal a feed which comprises a hemp plant or a part thereof, wherein the dairy animal is a dairy cow; obtaining milk from the dairy animal; and processing the milk into a storable milk product through at least one technical processing step. In certain embodiments the invention provides a pharmaceutical composition comprising a milk product that contains at least one cannabinoid active substance, wherein the milk product is produced according to the method just described. In certain further embodiments of the above-described method, the milk product is dehydrated and the technical processing step is selected from lyophilization, spray process drying, horizontal belt drying and fluidized bed drying. In certain other further embodiments the step of processing comprises a thermal treatment, which in certain still further embodiments comprises pasteurization. In certain other embodiments of the above-described methods, the feed comprises a hemp plant part comprising a hemp blossom of a pistillate hemp plant, and in certain other embodiments the feed comprises silage. In certain other embodiments of the above-described methods, the feed comprises 0.5 to 3% by weight of a hemp plant or a part thereof. In certain other embodiments of the above described methods the milk obtained from the dairy animal comprises 0.1-5 mg/l of Δ-9-tetrahydrocannabinol, and in certain embodiments the milk obtained from the dairy animal comprises 0.5-1 mg/l of Δ-9-tetrahydrocannabinol. In certain other embodiments of the above-described methods the storable milk product comprises yoghurt or cheese.

In another embodiment the present invention provides a method for producing a pharmaceutical product comprising an active cannabinoid substance, comprising feeding a dairy cow a feed which comprises a hemp plant or a part thereof; obtaining milk from the dairy cow; and processing the milk into a storable milk product through at least one technical processing step. In certain further embodiments the milk product is dehydrated and the technical processing step is selected from lyophilization, spray process drying, horizontal belt drying and fluidized bed drying. In certain other embodiments the step of processing comprises a thermal treatment, which in certain further embodiments comprises pasteurization.

These and other aspects of the present invention will become apparent upon reference to the following detailed description. All references disclosed herein are hereby incorporated by reference in their entireties as if each was incorporated individually.

### DETAILED DESCRIPTION OF THE INVENTION

The present invention derives from the unexpected observation that cows which have been fed at least in part with cannabis plants or parts thereof in the form of fresh fodder, dry fodder or silage fodder tolerate such feed and appear to feel absolutely comfortable, and the surprising discovery that such cows deliver a milk with an active substance (e.g., THC, Δ-9-tetrahydrocannabinol) content. The milk thus obtained can be further processed into milk products, such as ultra high temperature treated (UHT) milk, yoghurt or also cheese, having an active substance content to the extent the products obtained are within the framework of the laws of the jurisdiction of production and/or of consumption.

The present invention provides cannabinoid active substances in a form that is of natural origin, that is suitable for administration to individuals (e.g., human subjects or patients in need thereof) and that is innocuous for the consumers or patients. Furthermore the product can be largely produced on farms. As described herein, it has been found that milk from cows fed at least partially with hemp plants contains cannabinoid active substances and s therefore such milk, or products thereof, represents a form of administration of cannabinoid active substances which does not put too much strain on the patient.

The subject matter of the present invention thus relates to a method of producing milk and milk products which have a content of cannabinoid active substances and which are particularly suitable as a base for natural medicines, whereby milk cows are fed feed containing at least in part hemp plants or parts thereof, and the milk is obtained and further processed into a storable milk product through at least one technical processing step. Although the present description refers to preferred embodiments which comprise compositions and methods that relate to milk produced from dairy animals that are dairy cows (e.g., genus Bos), the invention is not necessarily intended to be so limited and in certain embodiments contemplates milk produced from other dairy animals that are known in the dairy arts, i.e., domesticated mammals such as goats, sheep, camels or other species from which milk may be obtained.

According to certain embodiments of the invention, the technical processing step is selected from among the following processing steps: lyophilization (e.g., freeze-drying), drying according to the spray process, horizontal belt drying and fluidized bed drying. A wholly dehydrated milk is thereby obtained with a content of cannabinoid substances. According to another embodiment of the invention, the milk obtained is further processed into a storable milk through a thermal treatment, such as pasteurization. According to another embodiment of the invention, hemp plants or hemp plant parts, in particular hemp blossoms of pistillate hemp plants, are used as feed admixture which have been subjected before their use to an ensilage to make them storable.

According to other embodiments of the invention, the cows are fed with a mixed fodder or concentrated fodder which, with hemp of good quality, contains approximately up to 3%, typically about 0.5 to 3% by weight of hemp components, while with lesser quality hemp, it can have a content of at least about 3 to 8% by weight, which typically according to certain preferred embodiments may be at least about 3%, 4%, 5%, 6%, 7%, 8% or more.

According to a further embodiment of the invention, a milk is obtained containing a Δ-9-tetrahydrocannabinol content of about 0.1-5 mg/l, preferably of about 0.5-1 mg/l, and in other preferred embodiments of at least about 1-2 mg/l, 2-3 mg/l, 3-4 mg/l, 4-5 mg/l or more of Δ-9-tetrahydrocannabinol. According to a further embodiment of the invention, the milk obtained is processed into yoghurt or cheese. According to a further embodiment of the invention, a storable milk product is used, which has been obtained according to the method of the present invention, for producing a pharmaceutical product having a content of cannabinoid active substances. For this purpose, methods are used which are common in production of natural medicines.

In the method according to the present invention, dairy animals such as cows (e.g., dairy cows, genus Bos) are fed with feed containing at least in part hemp plants or parts thereof. The hemp material is preferably material containing blossoms from the upper part of the plant. The harvest can be carried out with the same devices as for harvesting corn. Through the ensilage of the hemp material (e.g., preparation of hemp harvested while leaves are still green and storage under conditions that maintain succulence, such as by partial fermentation in a silo or the like, to produce silage) a material is obtained which has a better stability under storage conditions than dried hemp blossom material. The rest of the feed can be silage fodder, dry fodder or fresh fodder. Preferably the feed is mixed by a Total Mix Ratio (TMR) mixer in order to obtain a feed mixture having as uniform a content of the different components as possible. A milk is thereby obtained containing biologically active substances from hemp plants. The milk can be used as raw material for further processed medicinal products, stable under storage conditions, in particular natural medicines. An interesting product, for example, is wholly dehydrated milk comprising an active substance content such as that described herein. The Δ-9-tetrahydrocannabinol content can vary in the range of at least about 0.15 mg/l to 1 mg/l, and can be increased to at least about 5 mg/l under optimal conditions. The milk obtained can also contain other active substances which are not explicitly mentioned herein. The powdered milk obtained in this way can as such be made available as a pharmaceutical product. However, it is also possible and may in certain contemplated embodiments be desirable to use wholly dehydrated milk of this kind with other active ingredients as components of pharmaceutical products, where doing so makes sense from a therapeutic standpoint.

A preferred storable milk product that is stable under a wide range of storage conditions may comprise wholly dehydrated milk, which is obtained by treatment according to any of a number of technical processing steps known to those familiar with the art, for instance by freeze-drying the milk. Technical processing methods that may be employed according to certain preferred embodiments of the invention thus include lyophilization, drying according to the spray process, horizontal belt drying and fluidized bed drying. A powdered milk of this kind with an active substance content is an ideal raw material for the production of pharmaceutical compounds for use in the treatment of various medical conditions, including those described above. The invention also contemplates the use of other processing steps as known to the dairy arts, including, for example, thermal treatment steps which relate to controlled termperature regulation of milk in the course of processing, such as pasteurization and/or other procedures such as those used in preparing various milk, cheese and yoghurt products.

It is expressly stated that the products obtained according to the present invention are intended exclusively for use as permitted by applicable law.

The invention will be explained in the following with reference to an example, whereby this example should not represent any limitation.

### Example

A dairy cow with a medium milk production was fed over a time period of two weeks with a daily feed amount of 14 kg/day, divided up into two rations, 50 g of hemp blossoms having been added to each ration. The hemp blossoms used consisted of the upper part of pistillate hemp plants which were harvested in a way analogous to that employed for harvesting corn. The material was processed by a corn chaff cutter and underwent an ensilage step. The rest of the fodder was normal cattle feed (Total Mix Ratio, TMR) as is commonly provided in agriculture to cows for milk production. The content of Δ-9-tetrahydrocannabinol active substance in milk samples obtained one week after initiation of the feeding plan was determined to be 0.15 mg/l and 0.63 mg/l. The measurements were carried out in an accredited laboratory (Swiss testing STS 017, EN 45001, GMP).

In a trial, 20 kilograms of the milk obtained was freeze dried and processed into powdered milk. Here, too, it was noted by a group of test persons to whom the product was administered that the powdered milk obtained had a cannabinoid effect.

What is claimed is:

1. A method of producing a milk product that contains at least one cannabinoid active substance, comprising:
feeding a dairy animal a feed which comprises a hemp plant or a part thereof, wherein the dairy animal is a dairy cow;
obtaining milk from the dairy animal; and
processing the milk into a storable milk product through at least one technical processing step.

2. A pharmaceutical composition comprising a milk product that contains at least one cannabinoid active substance, wherein said milk product is produced according to the method of claim 1.

3. The method according to claim 1 wherein the milk product is dehydrated and wherein the technical processing step is selected from the group consisting of lyophilization, spray process drying, horizontal belt drying and fluidized bed drying.

4. The method according to claim 1 wherein the step of processing comprises a thermal treatment.

5. The method of claim 4 wherein the thermal treatment comprises pasteurization.

6. A method according to any one of claims 1 and 3-5 wherein the feed comprises a hemp plant part comprising a hemp blossom of a pistillate hemp plant.

7. A method according to any one of claims 1 and 3-5 wherein the feed comprises silage.

8. The method according to any one of claims 1 and 3-5 wherein the feed comprises 0.5 to 3% by weight of a hemp plant or a part thereof.

9. The method according to any one of claims 1 and 3-5 wherein the milk obtained from the dairy animal comprises 0.1-5 mg/l of Δ-9tetrahydrocannabinol.

10. The method according to any one of claims 1 and 3-5 wherein the milk obtained from the dairy animal comprises 0.5-1 mg/l of Δ-9tetrahydrocannabinol.

11. The method according to any one of claims 1 and 3-5 wherein the storable milk product comprises yoghurt or cheese.

12. A method for producing a pharmaceutical product comprising an active cannabinoid substance, comprising:
feeding a dairy cow a feed which comprises a hemp plant or a part thereof;
obtaining milk from the dairy cow; and
processing the milk into a storable milk product through at least one technical processing step.

13. The method according to claim 12 wherein the milk product is dehydrated and wherein the technical processing step is selected from the group consisting of lyophilization, spray process drying, horizontal belt drying and fluidized bed drying.

14. The method according to claim 12 wherein the step of processing comprises a thermal treatment.

15. The method of claim 14 wherein the thermal treatment comprises pasteurization.
